(19) United States
(12) Patent Application Publication
(10) Pub. No.: US 2006/0198530 A1
(43) Pub. Date: Sep. 7, 2006

(54) Hearing aid with automatic sound storage and corresponding method

(75) Inventors: Eghart Fischer, Schwabach (DE); Uwe Rass, Nurnberg (DE)

Correspondence Address: SCHIFF HARDIN, LLP;PATENT DEPARTMENT, 6600 SEARS TOWER, CHICAGO, IL, 60606-6473, US

(73) Assignee: Siemens Audiologische Technik GmbH

(21) Appl. No.: 11/365,402

(22) Filed: Mar. 1, 2006

(30) Foreign Application Priority Data
Mar. 2, 2005 (DE) 10 2005 009 530.5

(51) Int. Cl. H04R 29/00; A61B 5/12

(52) U.S. Cl. 381/60; 73/585

(57) ABSTRACT

The adaptation of a hearing aid to the wearer is made more convenient according to a hearing aid and method of use. A hearing situation is classified according to a hearing situation at any one time, the sound of the hearing aid is stored, and subsequently the sound setting with the assigned classification is stored. In this case, the sound setting is automatically stored with the associated classification after each adjustment.

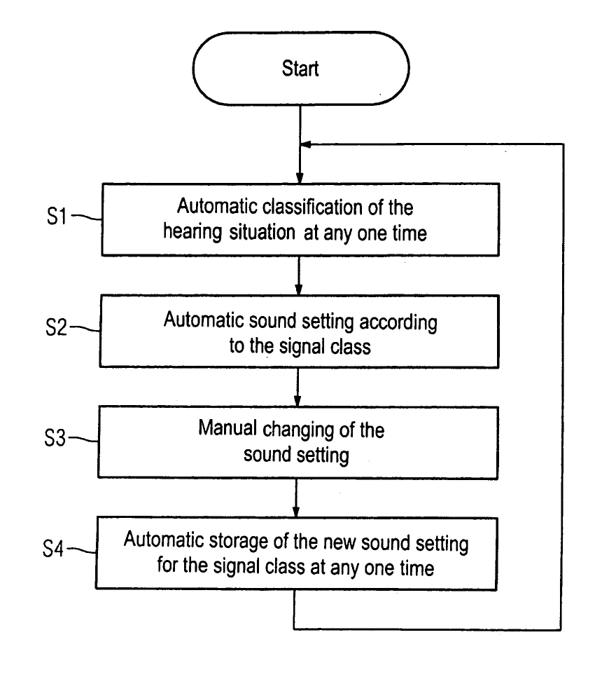

### BACKGROUND

The present invention relates to a hearing aid with a classification device for classifying a hearing situation at any one time, a setting device for setting the sound of the hearing aid, and a memory device, which is connected to the classification device and the setting device, for storing a sound setting with the assigned classification. In addition, the present invention relates to a corresponding method for operating a hearing aid.

The individually preferred sound settings of a hearing aid are dependent on the acoustic surroundings. For example, higher levels are desired for music at low frequencies than when holding a conversation in noisy surroundings. This means that the sound setting is generally carried out according to the signal class.

Modern hearing aids have classification systems which sense the hearing situation at any one time and perform corresponding settings in the hearing aid. These settings are determined during an adapting session with an acoustician and entered in the hearing aid as a fixed setting. From this fixed setting, the wearer of the hearing aid can then manually perform changes of the sound settings corresponding to changed acoustic requirements in each case from the fixed setting for the respective hearing situation. To avoid constant resetting, readjustment with the acoustician is necessary.

European Patent EP 0 814 634 B1 discloses a programmable hearing aid system. In this case, the set of parameters of a programmable hearing aid assigned to each hearing situation is not already established during adaptation with the hearing aid acoustician, but instead a number of test parameter sets are provided at the beginning for each hearing situation. In an optimizing phase, the wearer of the hearing aid can then determine which set of parameters is individually best suited for him in the individual hearing situations. The set of parameters is finally permanently assigned to the hearing situation. This constitutes adaptation to actual acoustic surrounding conditions, which can largely be performed by the wearer of the hearing aid himself.

In addition, the German patent document DE 101 52 197 A1 describes a method for programming a hearing aid with the aid of a remote control. The wearer of the hearing aid can switch his hearing aid to a receiving mode, in which absolute or relative parameter changes concerning the volume, high- or low-frequency boosting, activation of a noise filter, etc., are recorded. Recording is ended by actuating a memory button and stored in the hearing aid or the remote control. If the hearing aid then detects the same hearing situation once again, not only the setting for the hearing program provided for this but also the previously recorded operating steps become effective, so that an individual adaptation of the hearing program to the respective surrounding situation takes place.

The European patent document EP 1 453 356 A2 also discloses a method for setting a hearing system, it being possible for interactive adaptation to be performed during operation. A classifier detects different hearing situations and starts interactive adapting procedures, within which various settings must be assessed. In this case, only settings which match the hearing situation are offered.

### SUMMARY

The object of the present invention is to make the individual adaptation of a hearing aid more convenient.

This object is achieved according to various embodiments of the invention by a hearing aid with a classification device for classifying a hearing situation at any one time, a setting device for setting the sound of the hearing aid, and a storage device, which is connected to the classification device and the setting device, for storing a sound setting with the assigned classification, it being possible for a sound setting to be automatically stored in the storage device with the associated classification after each setting.

Also provided according to various embodiments of the invention is a method for operating a hearing aid by classifying a hearing situation at any one time, setting the sound of the hearing aid, and storing a sound setting with the assigned classification, a sound setting being automatically stored with the associated classification after each adjustment.

Such a hearing aid is therefore constantly in a learning mode, the last changes/settings performed by the user always being stored in the latest hearing program. The settings are then taken over once again when the hearing aid is switched again into this program.

The setting device preferably comprises a remote control. This ensures more convenient operation of the hearing aid. With the remote control, the sound of the hearing aid can preferably also be set for the hearing situation at any one time.

According to a preferred embodiment, the classification device can be used to switch automatically into a signal class corresponding to the hearing situation at any one time, with the associated sound setting. With the automatic storage of the sound setting, it is not only retained in the last chosen state when the hearing aid is switched on and off but also when there is an automatic change of the hearing program according to the respective signal class.

With the automatic switching-over of the hearing program, in a preferred embodiment, there is cross-fading from a first sound setting into a second sound setting. This has the advantage that the wearer of the hearing aid is not surprised by a high volume if a specific frequency band in the newly switched-on hearing program is amplified to a much higher level than in the old hearing program.

### DESCRIPTION OF THE DRAWINGS

The present invention is now explained in more detail on the basis of the accompanying drawing, which shows a flowchart of the way in which the hearing aid is set according to an embodiment of the invention.

### DETAILED DESCRIPTION OF THE PREFERRED EMBODIMENTS

The exemplary embodiment described below represents a preferred embodiment of the present invention. According to the flowchart represented in the figure, after switching on the hearing aid or starting, an automatic classification of the hearing situation at any one time takes place, corresponding to step S1, with the classification device of the hearing aid. The hearing aid uses this classification information to load the sound setting corresponding to the classification information automatically from a memory, as indicated in step S2.

The hearing aid system therefore has a setting device which makes it possible to set the sound during day-to-day operation. In the present example, the setting device is realized by a remote control, although any know control mechanism may be utilized. The wearer of the hearing aid therefore uses the buttons on the remote control for the sound setting. This sends information on the adjustment of the sound controller wirelessly to the hearing aid.

In the hearing aid, the hearing situation at any one time has already been sensed with the aid of the classifier, corresponding to step S1, so that the change in sound can be assigned to the detected class. The new sound setting is then stored in the memory, corresponding to step S4, for the signal class at any one time.

The memory of the hearing aid contains a number of sound settings for each signal class and for each hearing aid program. If the hearing situation changes, the corresponding sound setting is automatically called up from the memory and set, corresponding to S1 to S4. The transition of the sound setting takes place by slow cross-fading.

To make the setting of a multi-program hearing aid even more convenient, the sound settings are synchronized in a suitable way between the hearing programs. In other words, a setting for speech in quiet conditions, for example, is transferred into a fixed program for quiet surroundings in an automatic program. Consequently, the wearer of the hearing aid does not have to carry out the same sound setting in all hearing programs for similar hearing situations.

It is consequently ensured according to the invention that the wearer of the hearing aid can conveniently adjust the sound of his aid and adapt it to the hearing situation at any one time. The hearing aid then stores the settings automatically, so that the wearer of the hearing aid does not have to perform the settings again after switching over or switching off the aid. The hearing aid therefore always remembers the respective setting and is virtually always in a learning mode. It goes without saying that the hearing aid may also be provided with the option of switching off the learning mode.

According to the invention, it is consequently possible for long-term acclimatization effects, changing typical acoustic surroundings and changing hearing loss of the user to be allowed for in the settings by the user himself, without having to visit the acoustician.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A hearing aid, comprising:
a classification device for assigning a classification to a hearing situation at any one time;
a setting device for setting the sound of the hearing aid; and
a memory device, which is connected to the classification device and the setting device, for storing a sound setting with the assigned classification, permitting a sound setting to be automatically stored in the memory device with the assigned classification after each setting.

2. The hearing aid as claimed in claim 1, wherein the setting device comprises a remote control.

3. The hearing aid as claimed in claim 2, wherein the remote control comprises a setting mechanism permitting the remote control to set hearing aid sound for the hearing situation at any one time.

4. The hearing aid as claimed in claim 1, wherein the classification device comprises a mechanism to switch automatically into a signal class corresponding to a hearing situation at any one time, with the associated sound setting.

5. The hearing aid as claimed in claim 4, comprising a cross-fading mechanism for cross-fading from a first sound setting into a second sound setting.

6. A method for operating a hearing aid, comprising:
assigning a classification to a hearing situation at any one time;
setting a sound of the hearing aid; and
automatically storing the sound setting with the assigned classification after each said setting.

7. The method as claimed in claim 6, further comprising:
automatically switching into a signal class corresponding to a hearing situation at any one time with the associated sound setting.

8. The method as claimed in claim 7, further comprising:
cross-fading from a first sound setting into a second sound setting.

9. The method as claimed in claim 6, further comprising: automatically storing a sound setting for a first hearing program for the hearing situation at any one time in a similar way for a second hearing program.
